(19) United States
(12) Patent Application Publication
(10) Pub. No.: US 2012/0302875 A1
(43) Pub. Date: Nov. 29, 2012

(54) SYSTEM AND METHOD FOR INSERTING INTRACRANIAL CATHETERS

(76) Inventor: Gregory Allen Kohring, Hennef (DE)

(21) Appl. No.: 13/569,191

(22) Filed: Aug. 8, 2012

(51) Int. Cl. A61B 1/04; A61B 8/12

(52) U.S. Cl. 600/424

(57) ABSTRACT

An improved system for safely and accurately placing intracranial catheters by using techniques from the field of artificial intelligence (AI) which combine the output from in vivo ultrasonic sensors with in vivo video cameras and an embedded inertial measurement unit. The AI subsystem synthesis the output from the three sensors to determine an optimal route to the desired intracranial site while avoiding larger blood vessels en route. Additionally, by using the output from the inertial measurement unit, the catheter's complete trajectory can be recorded and made available for post-operative analysis. The entire system is portable so that it can be used outside of the hospital operating room, for example, in an intensive care unit. Compared to standard freehand methods of placing intracranial catheters, the system embodied here will reduce concomitant hemorrhaging while increasing the accuracy of catheter placement.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The field of the present invention relates to those systems, methods and devices for accurately and safely inserting a catheter into a body. In particular the system, method and apparatus described herein supports the intracranial insertion of catheters without the attendant complications and disadvantages of known technologies.

2. Description of Related Art

In the field of neurosurgery it is often required to insert devices of various type into the brain. When the device is tubular in shape it is generally referred to as a catheter. Catheters allow the drainage or administration of fluids as well as the placement of medical probes and sensors. A Catheter may be stiff or flexible depending upon the intended usage.

A ventriculostomy is one particular neurosurgical procedure whereby a catheter is inserted through an opening in the skull and into one of the ventricles of the brain in order to drain excess cerebrospinal fluid (CSF). When the fluid build up is due to disease or congenital factors the catheter may need to remain in place for a long period and is often referred to as a shunt, because the fluid is redirected back into the body through the peritoneum. When the fluid build up is due to severe brain trauma (e.g., a blow to the head), then the catheter will only remain in place as long as needed and is referred to as an External Ventricular Drain (EVD).

Inserting a catheter into a ventricle of the brain is a delicate procedure due to the risks of collateral brain damage. Therefore when inserting a shunt, the standard practice calls for CT (Computed Tomography) scans to be used in the careful planning of a route which will, to the fullest extent possible, avoid damaging sensitive areas of the brain as well as limiting concomitant hemorrhaging. The CT images may further be fed into an image guided, microsurgery robotic system (e.g., the robot described in U.S. Pat. No. 7,155,316) for full automatic route planning and execution. A drawback of robotic systems is that they tend to be very bulky, taking up quite a bit of floor space. For this reason they are to be found mostly in a larger OP (Operating Room).

For the case of trauma patients, pressure within the ventricles can rise to life threatening levels within a short time. Under such circumstances, the attending physician does not have an opportunity to carefully plan the optimal route to the ventricle, rather the EVD must be inserted with minimal delay while the patient is in an ICU (Intensive Care Unit) not in the OP. The standard procedure in this case calls for the physician to create an opening in the skull, called a burr hole, then to insert the catheter by holding it in the most likely direction of the ventricle and pushing it inward with the hands. The freehand insertion procedure is not very accurate with studies (Huyette, et al., 2008) showing that freehand placement typically required two attempts before the EVD was satisfactorily placed and CSF was flowing. Standard hospital procedures generally call for no more than four attempts to place an EVD freehand, before resorting to other solutions.

Once the patient's condition has stabilized, CT scans can be performed to estimate EVD related damage. In one such study (Gardner, et al. 2009), it was revealed that 33% of freehand placed EVDs were definitely linked to hemorrhagic complications and 2.5% likely linked to permanent brain damage. (Deaths directly attributable to the procedure are difficult to ascertain as patients are usually in a very critical condition to begin with and CT scans are rarely performed after a patient has died.)

For determining the amount of EVD related damage using CT scans the catheter must remain in place until the scan is taken. This has several disadvantages: First, extended durations of EVD placement have been implicated as a risk factor in EVD related infections (Kim, et al., 2012). Second, if several attempts are made to sit the EVD or the EVD must be revised because the CSF flow is week, then only the final catheter placement will be visible on the CT scan; information regarding the position of the previous placements is lost.

Catheter guides external to the skull (U.S. Pat. No. 4,613,324, U.S. Pat. No. 6,206,885, U.S. Pat. No. 7,122,038 and US 2010/0036391 A1) increase the accuracy of placement by holding the catheter in the most probable direction of the ventricles based upon external anatomical landmarks. However, the exact location of the ventricles within the brain depends upon patient specific factors that cannot be estimated using external information alone.

Precise positioning along with the avoidance of hemorrhagic complications requires intracranial imaging capabilities. In one such system (US 2007/0083100 A1) an ultrasound transducer was built into the tip of the catheter. The ultrasound image was then displayed on a computer screen. Ultrasound acting as a far field sensor is able to locate the ventricles thereby guiding the catheter in the right direction. Simultaneously to far field sensing precise near field sensing is required as arteries having a diameter of 0.5 mm can lead to significant hemorrhaging if damaged.

An intracranial imaging system using a combination of an ultrasonic transducer and a fiber optic lens has also been suggested (U.S. Pat. No. 5,690,117). However, displaying the image from the fiber optic lens on a computer screen requires an external device to convert the optical signal into an electronic signal thereby adding complexity and costs to the overall system. Using a solid state imaging device embedded in the catheter's distal end decreases the complexity of the system (U.S. Pat. No. 5,989,185; U.S. Pat. No. 5,325,847; U.S. Pat. No. 5,305,736), although, simply displaying both images on the screen does not take advantage the synergies possible with the use of multiple sensors of different modality.

If, by consulting the optical image, the physician would notice than an artery is in the catheter's path and if the physician were to adjust the path of the catheter accordingly, then this path change needs to be recorded. One method for tracking the location of a probe inside a body is to include an electro-magnetic device in the probe tip that emits a signal which can be detected outside the body using a special detection device (U.S. Pat. No. 4,431,005). Such approaches are clumsy in practice since they require a second device to be used simultaneously while the physician is inserting the catheter. External ultrasound scanners on the other hand employ solid state gyroscopes and accelerometers to determine the position of the scanner on the surface of the body (U.S. Pat. No. 6,122,538). As noted above, once the patient's condition has stabilized, CT scans will be performed to determine the full extent of the injuries. If the exact trajectory of the catheter is known, then this trajectory can be superimposed upon the CT scans to determine to what extent the ventriculostomy itself resulted in additional damage. To determine the full trajectory requires tracking the position of the catheter with respect to the burr hole using three dimensional stereotactic coordinates.

Once the attending physician has started a ventriculostomy they will attempt to insert the catheter at a constant rate while holding it steady. Under these circumstances the physician's attention is focused on the patient and not the computer display. None of the previous inventions have used computer aided sensor processing to automatically warn the physician if the catheter is off course, has passed through the ventricle or is in danger of intersecting an artery. Techniques for recognizing blood vessels and other anatomical structures in medical images have been discussed in the literature (U.S. Pat. No. 6,217,519, U.S. Pat. No. 6,956,602; US 2010/0054525 A1; US 2010/0135561 A1; Liu, et al. 2007; Almoussa, et al. 2011), as have techniques for recognizing tissue structures in endoscopic images (US 2007/0015989 A1) and techniques for recognizing tissue boundaries in ultrasound images (U.S. Pat. No. 8,050,478 B2; U.S. Pat. No. 5,457,754; Avianto and Ito, 2000).

In summary, the heretofore disclosed solutions to the long standing problem of accurately and safely performing a ventriculostomy on trauma patients in an ICU environment suffer from a number of disadvantages: a) they lack an automated system capable of taking advantage of the synergisms arising through the combination of different sensor input in order to provide an intelligent navigation aid for the physician, b) they lack a means to minimize concomitant hemorrhaging, c) they lack a means to record the trajectories of the catheter, thereby hampering post-operative diagnostics, and d) to the extent that robotic systems are available, they do not integrate well into an ICU environment, requiring instead the use of an OP.

### BRIEF SUMMARY OF THE INVENTION

The system constructed in accordance with one embodiment utilizes a video camera in the distal end of the catheter along with an ultrasonic transducer. Embedded into the proximal end of the said catheter is an inertial measurement unit (IMU) comprising a gyroscope, a compass and a triaxial accelerometer. The output from all devices are passed through to a portable computer where they are analyzed using software which automatically detects whether or not the catheter is on course to intersect the ventricle, automatically detects blood vessels lying in the catheter's path, automatically plans a route to minimize hemorrhaging and then employs augmented reality techniques to display the best route overlayed on the real time video output. Furthermore, the software calculates and records the position of the catheter relative to the burr hole during the entire procedure and makes this information available for post-operative diagnostics.

Accordingly there are several advantages of one or more aspects: a) the in vivo video camera decreases system complexity; b) the IMU allows the software to record the complete trajectory of the catheter for post operative analysis and should a revision be necessary, the new trajectory, together with the old trajectory are available for post-operative analysis; c) the software intelligently combines the data from the ultrasound sensor, video camera and IMU to detect blood vessels, monitor the catheter's trajectory with respect to the location of the ventricles, and suggests course corrections to avoid damaging blood vessels while remaining on target; and d) the entire system is portable to conform to ICU environments. Other advantages of one or more aspects will be apparent from a consideration of the drawings and ensuing description.

### BRIEF DESCRIPTION OF THE SEVERAL VIEWS OF THE DRAWINGS

FIG. 1 shows a schematic providing an overview;

FIG. 2 depicts a catheter with its distal and proximal ends;

FIG. 3 provides an enlarged view of the distal end of the catheter;

FIG. 4 provides an enlarged view of the proximal end as it connects to the body of the catheter;

FIG. 5 is a schematic showing a cross-section through the proximal end of the catheter;

FIG. 6 is a schematic showing a longitudinal cross-section through the proximal end of the catheter;

FIG. 7 presents a process diagram describing how the system is used;

FIG. 8 presents a flow diagram describing a means for implementing an intelligent navigation system;

FIG. 9 presents a flow diagram describing the computer assisted means for recording the catheter's trajectory;

FIG. 10 shows a cross-section through the proximal end of the catheter in an alternative embodiment; and

FIG. 11 shows a longitudinal cross-section through the proximal end of the catheter in an alternative embodiment.

### DETAILED DESCRIPTION OF THE INVENTION

### Structural

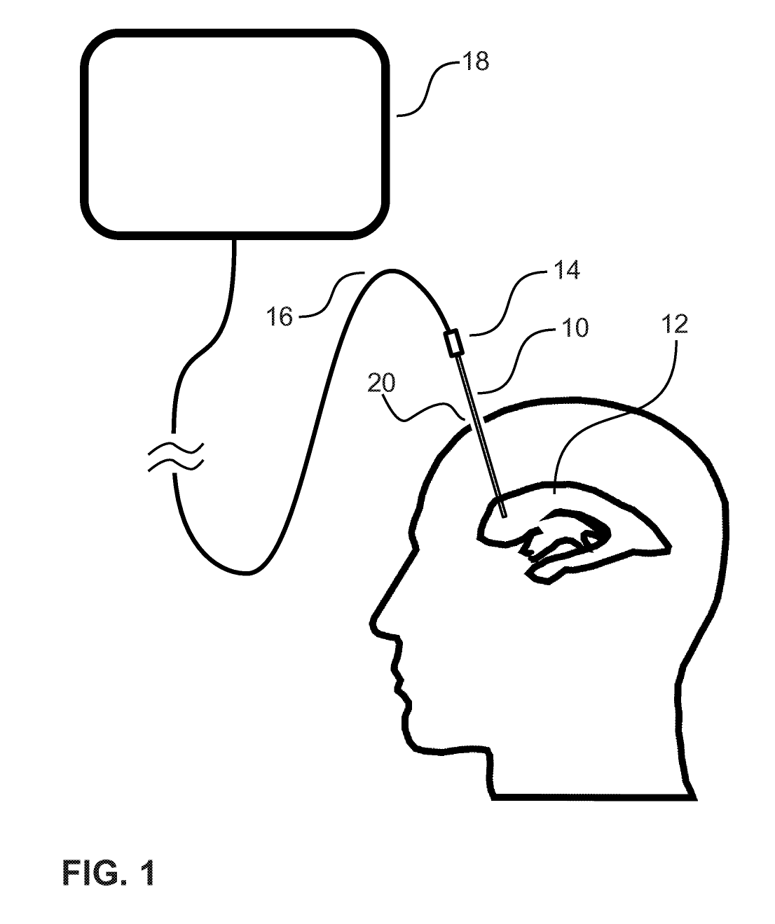

In FIG. 1 a catheter 10 is shown after having been inserted through a burr hole 20 and into the cerebral ventricle 12. The proximal end of the catheter 14 is communicatively coupled 16 with a portable computer 18. The computer 18 provides a means for acquiring and analyzing the data produced by the sensors, as well as a means for employing augmented reality in support of a means for providing intelligent navigation to the desired site in the body. In the first embodiment, the portable computer 18 is a high performance tablet PC, e.g., from the Hewlett-Packard Company of California. However, any portable computer with sufficient processing power to support the data analysis described below, may be substituted. In the first embodiment the communicative coupling 16 between the catheter and the computer 18 transpires over a standard USB interface.

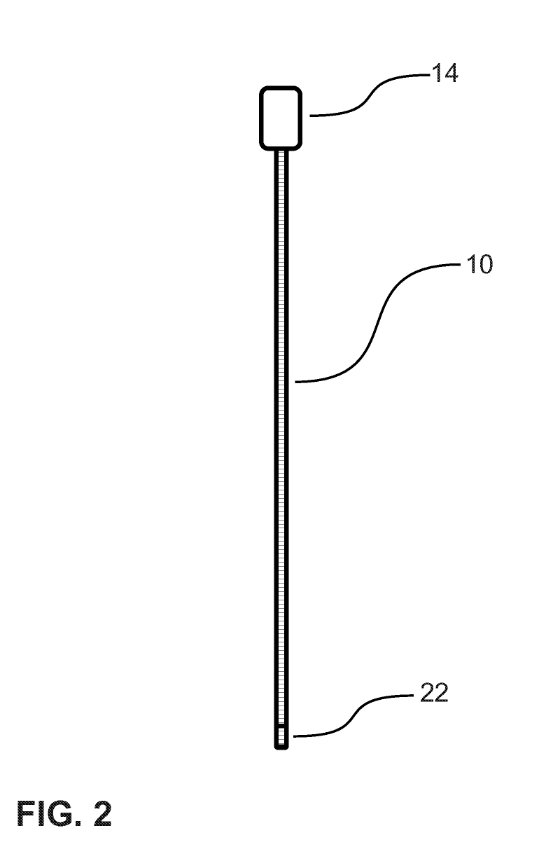

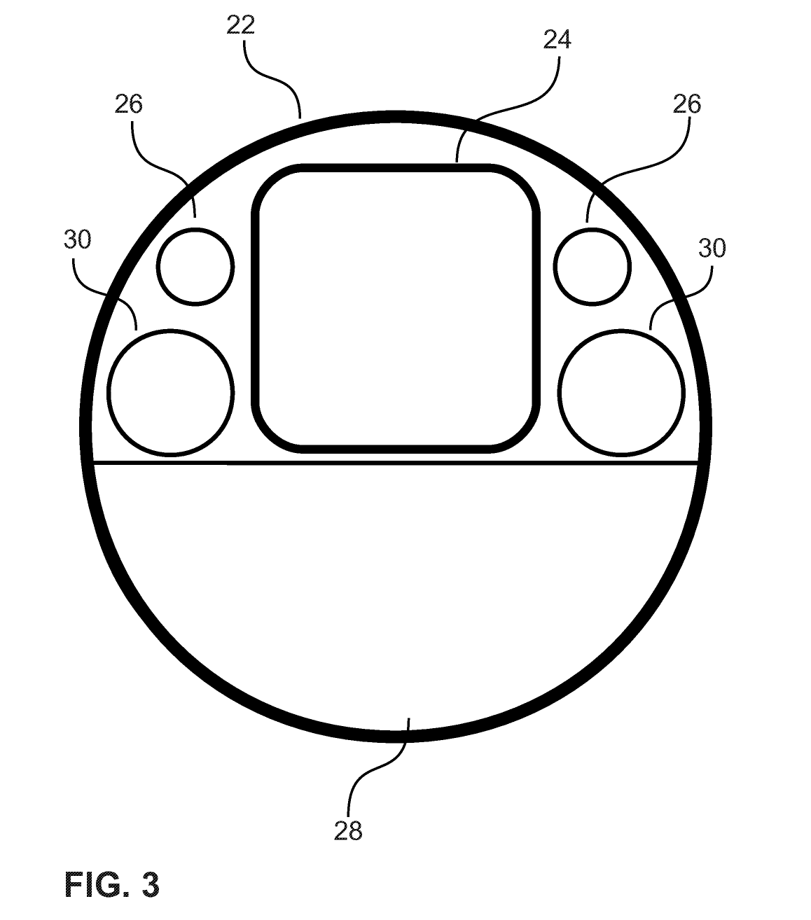

FIG. 2 shows the catheter 10 drawn to scale. In the first embodiment, the catheter 10 has an inner diameter of 2.5 mm and is manufactured out of a, surgically safe material, e.g., polyurethane. If the material out of which the catheter 10 is manufactured is not sufficiently stiff to allow it to pushed into the brain's soft matter, then a metal stylus can be inserted in the drain 28 (see FIG. 3) to provide the required stiffness. The stylus can be removed once the insertion procedure is over to allow the CSF to flow.

The catheter 10 has equally spaced markings running from the distal end to the proximal end. As will be explained later, these provide a means for determining the depth of insertion of the catheter 10, i.e., the distance of the catheter's distal end 22 from the burr hole 20. In the first embodiment it is contemplated to put depth markings every millimeter, but other intervals as well as other means of determining the depth of insertion are possible.

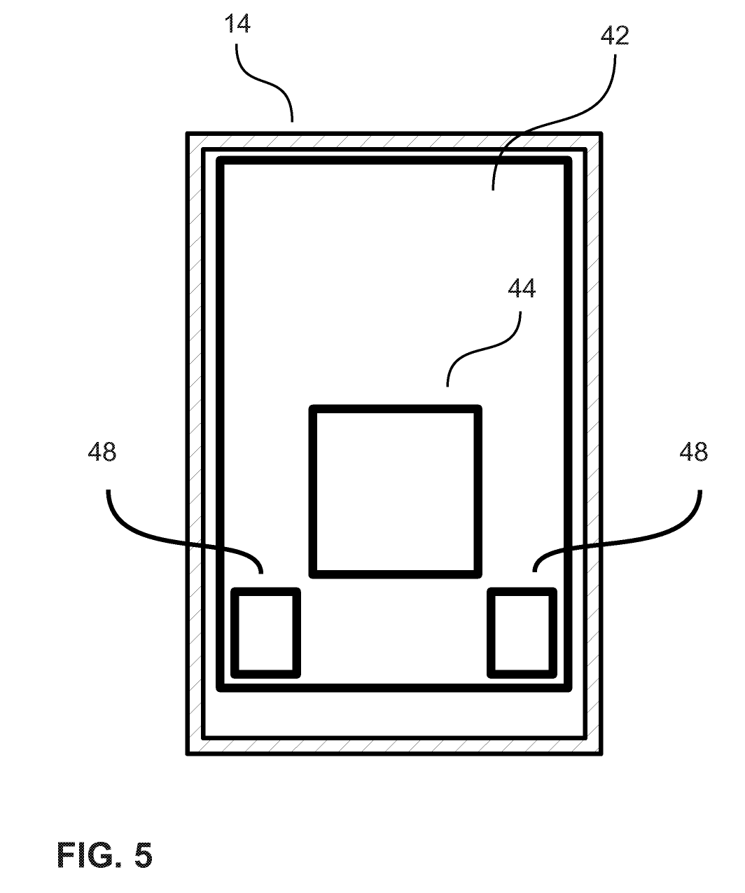

Built into the distal end 22 of the catheter 10 are multiple sensors. Located at the proximal end 14 of the catheter 10 is an IMU 44 (see FIG. 5) for use in determining the position of the catheter's distal end 22 with respect to a three dimensional stereotactic coordinate system centered on the burr hole 20.

In FIG. 3 a front view of the catheter's distal end 22 is presented showing one arrangement of the various components in the first embodiment. The catheter 10 has two main compartments, an upper compartment containing the electronic sensors and a lower compartment for draining the fluid. A video camera 24 is used as a short range sensor to locate blood vessels and other anatomical structures in the immediate vicinity. The first embodiment employs a full color video camera, e.g., the video camera from AWAIBA, Lda of Portugal with dimensions 1.1×1.1×2.2 mm. However, in other embodiments any video camera of similar size can be used.

Operating a video camera 24 in an intracranial space requires a means for illuminating a vicinity of the distal end 22. In the first embodiment, it is contemplated to use two fiber optic cable bundles 26 located on either of side of the camera, but other means for illuminating a vicinity of the distal may suffice.

Ultrasonic transducers 30 are located to the sides the camera. In the first embodiment the components needed to build a transducer of the required size can be obtained from the company, Boston Piezo-Optics, Inc. of Massachusetts. However, suitable components can be obtained from other companies as well. The ultrasonic transducers 30, comprises a means for locating a site in a body which cannot be identified in the video feed because it lies behind an opaque structure in front of the camera.

Finally, in the lower compartment, a drainage canal 28 is provided. In the first embodiment, the drainage canals occupy the space not need by the other components. In order to facilitate drainage, it is advisable to have openings in the wall of the catheter as well. Obviously, these openings should be on the side of the catheter where the drain is located.

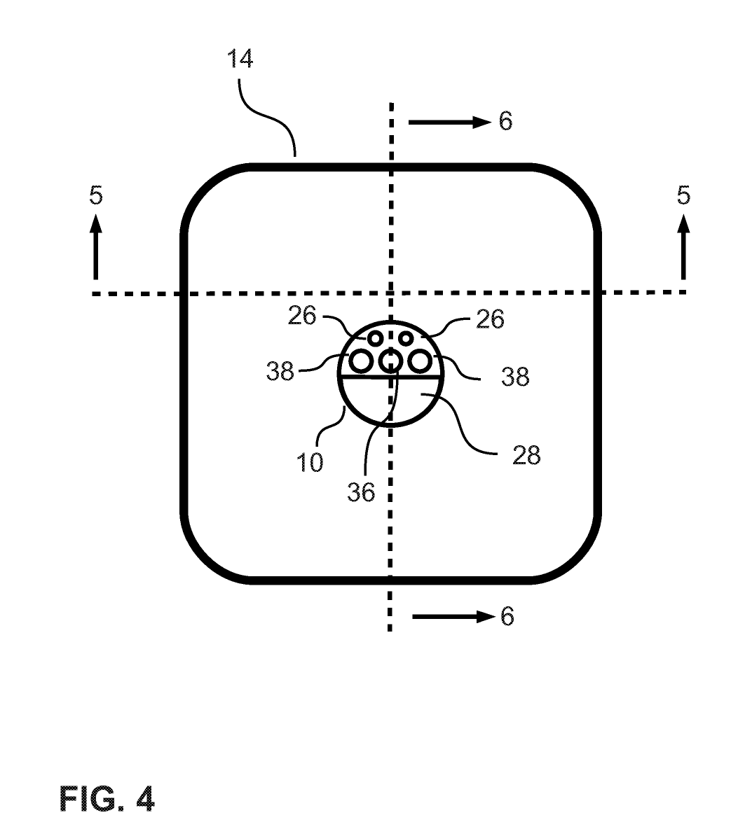

The distal end of the catheter 22 described above is covered with a transparent plastic end piece that prevents bodily fluids from coming in contact with the electronic parts, but has openings to allow fluid to flow into the drainage tubes. All necessary wiring and tubing run up the length of the catheter 10 to the proximal end, where the catheter 10 connects to the cap 14 as shown in FIG. 4. The wiring for each sensor in the first embodiment is clearly marked, with one bundle 36 for the video camera and two bundles 38 for the ultrasonic transducers. The fiber optic cables in the first embodiment 26 are also shown as is the tubing for the drainage canals 28.

FIG. 5 provides a cross-sectional view of the cap 14 showing the circuit board 42 containing an IMU 44, and the white light LED chips 48. An IMU is a composition of three complementary sensors: a gyroscope, a compass and a triaxial accelerometer. The compass is used to determine the direction in which the catheter 10 is pointing. The gyroscope is used to determine the angular orientation of the catheter 10 with respect to this direction. The accelerometer is used to determine the motion of the catheter 10 with respect to Earth's gravitational field. In the first embodiment, all three sensors are found on a single chip, e.g., the MPU-9150 from InvenSense, Inc. of California. However, other embodiments might use a single chip for each of the three sensors. The white light led chips 48, are commodity parts and can be obtained from a number of manufactures.

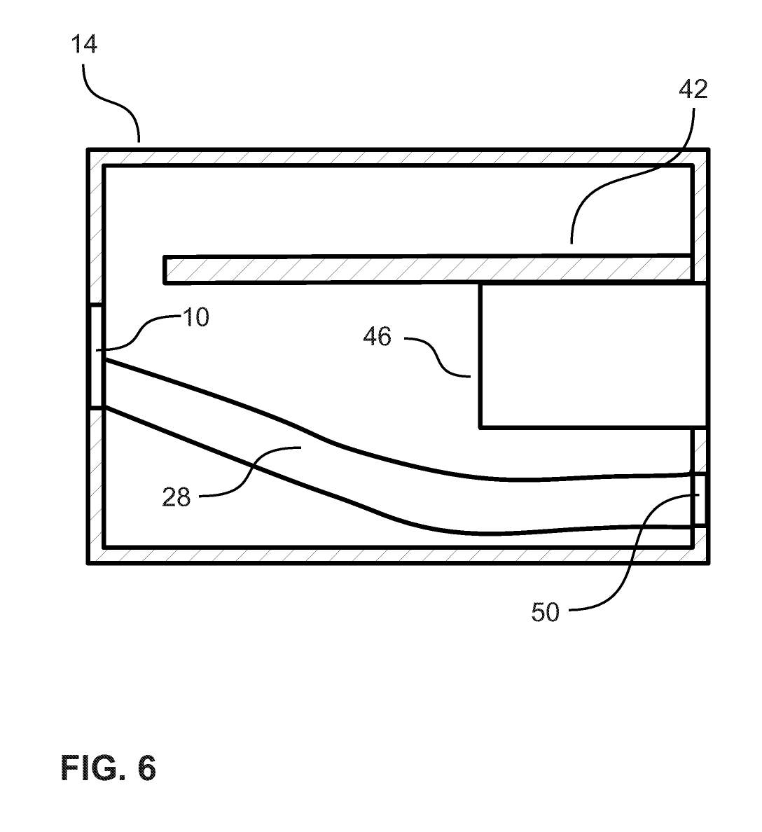

FIG. 6 shows a longitudinal cross-sectional view of the cap 14 in the first embodiment. The drainage tube 28, connects via a standard size opening 50 to a tube leading to the external CSF reservoir (not shown). Also shown is the placement of the micro B USB receptacle 46, which provides a plug for a USB cable connecting the catheter 10 with the computer 18. A USB cable provides not only the communicative coupling to the computer, but also the power to run the sensors.

The description presented herein should not be interpreted as precluding the further incorporation of passive sensors, i.e., sensors unconnected to the purpose of providing an intelligent navigational aid. For example, a pressure sensor like the FOP-125 from FISO Technologies, Inc. of Canada, which is only 125 microns in diameter could easily be incorporated without any major changes to the catheter design presented above.

### Operational

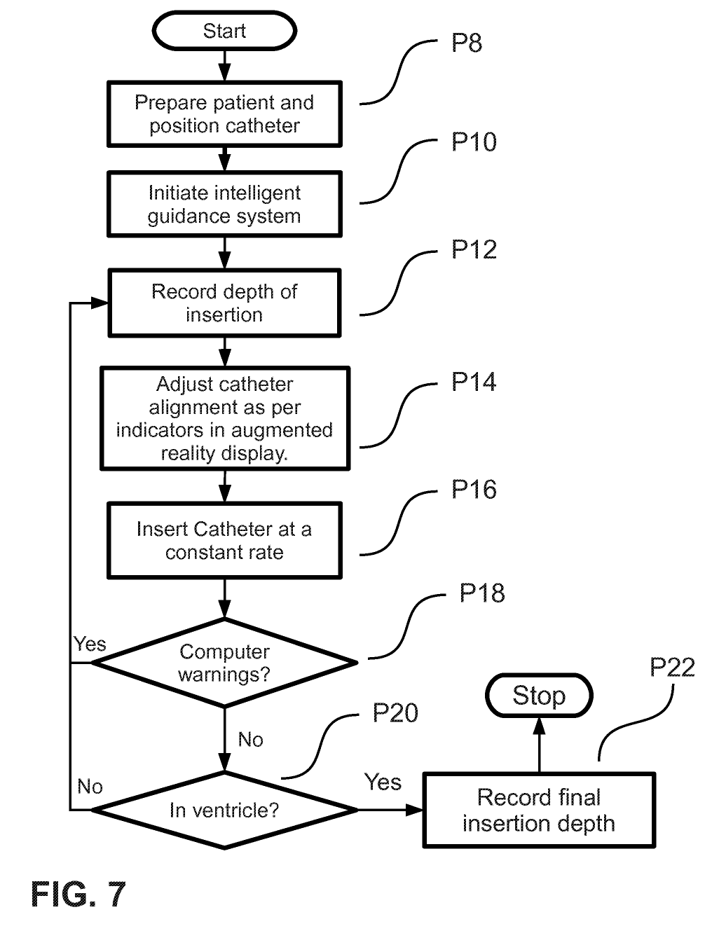

A procedure for using the device described above is, in the first embodiment, outlined in FIG. 7. First the patient is prepared according to the standard medical practice P8, after which the catheter 10 is brought into position by bringing the distal end 22 to the burr hole 10. A means for identifying the target intracranial site of the catheter's distal end 22 is provided by displaying the output from the ultrasound sensor on the computer screen 18. The catheter 10 can then be aligned it to point in the direction of the desired ventricle 12. The operator then initiates the intelligent guidance system P10 and records the initial depth P12 of the catheter by reading the millimeter markings on the catheter and inputting this information into the computer 18. Before proceeding the operator again checks the alignment P14. Now the operator proceeds to push the catheter in at a constant rate P16 until the computer issues an indicator P18. In the first embodiment both audio and visual indicators will be used. However, the modality of the indicator is not as important so long as the operator recognizes it and responds appropriately. What is to be indicated is one of the following situations: a) blood vessels are in the catheter's path, b) the catheter 10 is no longer pointed in the direction of a ventricle 12, or c) the catheter 10 has already reached a ventricle 12. In both a) and b) the operator returns to a previous step P12 and continues once again. In case c) the operator checks that the catheter has indeed reached the ventricle P20, then reads the final depth of the catheter 10 from the millimeter markings and inserts this information into the computer. The catheter 10 is now properly seated.

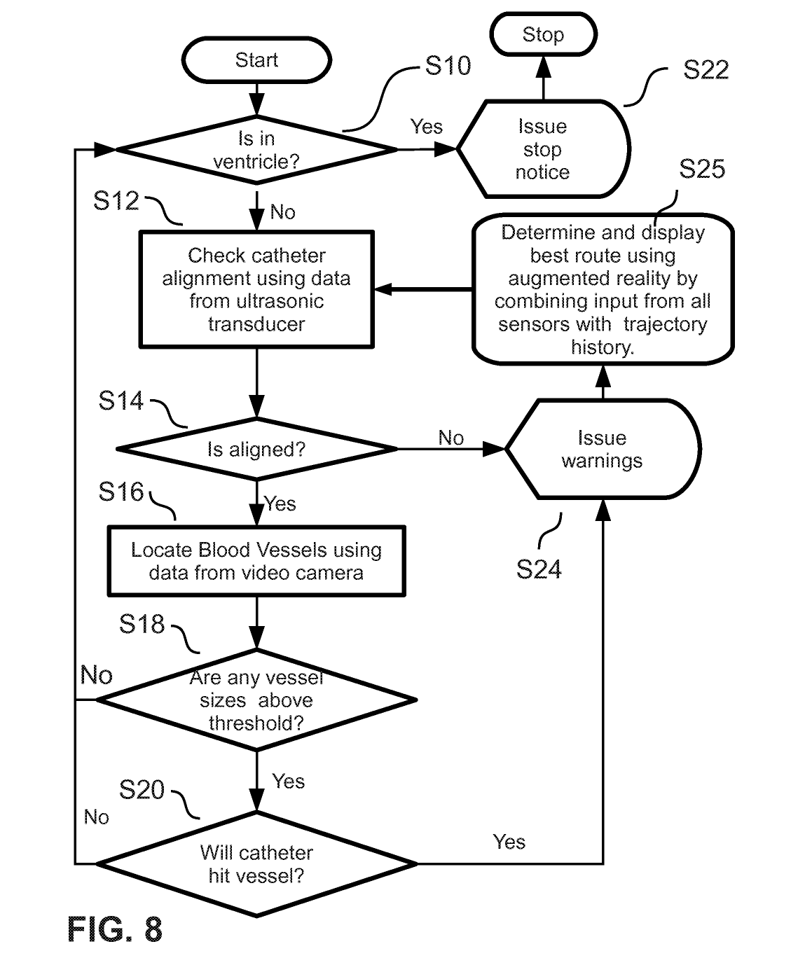

A high level flow diagram of the procedure which constitutes a means for providing intelligent navigation assistance to reach the desired site is shown in FIG. 8. The first step S10, checks the momentary location and alignment of the catheter's distal end 22. In particular, the algorithm uses anatomical clues in the images obtained from the video camera 24 to determine whether or not the catheter 10 has entered the ventricle 12. If so, then a stop notice S22 is issued. In the first embodiment it is contemplated that the warning is converted into an audio signal, but a visual cue may be suitable as well. Upon hearing the audio signal, the operator should proceed as for P20 described above. If the distal end of the catheter 22 is not in the ventricle, then the catheter's alignment is checked using the far field sensor input S12. If the catheter 10 is no longer aligned with the ventricle S14 then an audio signal will be issued S24, and the program will proceed to determine the how the catheter should be moved so as be brought into proper alignment. The position for proper alignment will be overlayed on the image from the video camera 24 thereby enabling the operator to quickly see what adjustments are necessary before proceeding. The technique for overlaying computer generated information with real time video input is generally known as augmented reality.

The next step for providing intelligent navigation assistance is to locate the blood vessels in front of the catheter 10 using the input from the video camera S16. In the first embodiment it is contemplated to use the algorithm of Liu and Zhang, but other algorithms may be suitable as well. If the blood vessel is below a critical size, then it is ignored S18. The critical size is an adjustable parameter set by the operator before the surgery is started. The standard value is expected to be 0.5 mm.

Once a blood vessel is located it must be determined whether or not the blood vessel lies directly in the path of the catheter S20. If the catheter 10 will not hit the blood vessel then it can be ignored, otherwise an indicator is issued S24 and the program will proceed to calculate a trajectory correction which avoids the blood vessel, but still keeps the catheter 10 on course to hit the ventricle 12. Again, using augmented reality S25, the position for proper alignment will be overlayed on the image from the video camera 24 thereby enabling the operator to immediately see what adjustments are necessary before proceeding.

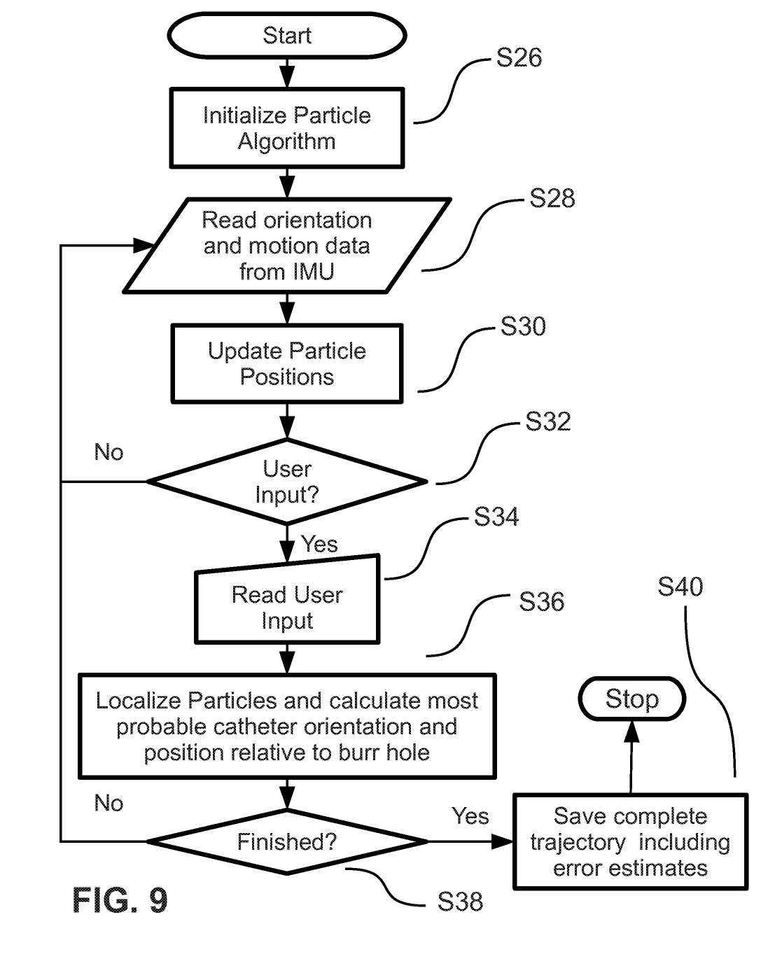

In order to calculate acceptable trajectory changes, a means for determining the local position of the catheter 10 relative to a reference point on the body is required. In the first embodiment, it is contemplated to use the so-called Particle Algorithm for determining the catheter's location. However, those skilled in the art will know that other techniques such as Kalman Filters may also be used. An outline of the algorithm for estimating the current position makes use of the information from the IMU 44, as shown in FIG. 9. After initializing the algorithm S26, the data is read S28 from the IMU 44 and the position of the particles is updated S30. As noted previously, the operator may occasionally record the catheter depth P12 and P22. If this information is available S32, it is read S34 and used to localize the particles S36 according to the common procedure of the particle algorithm. Once the ventricle 12 has been reached, the procedure is finished P20 and the algorithm finishes S38 by activating the means for storing the complete trajectory S40.

The particle algorithm will determine the location of the proximal end of the catheter 14 with respect to the burr hole. As the proximal and distal ends are connected by the rigid body of the catheter 10, those skilled in the art will recognize that the location of the distal end can be determined by simple geometry. Finally, for post-operative comparison with images from a CT scan, the burr hole 20 needs to be locatable on the CT images.

### Alternative Embodiments

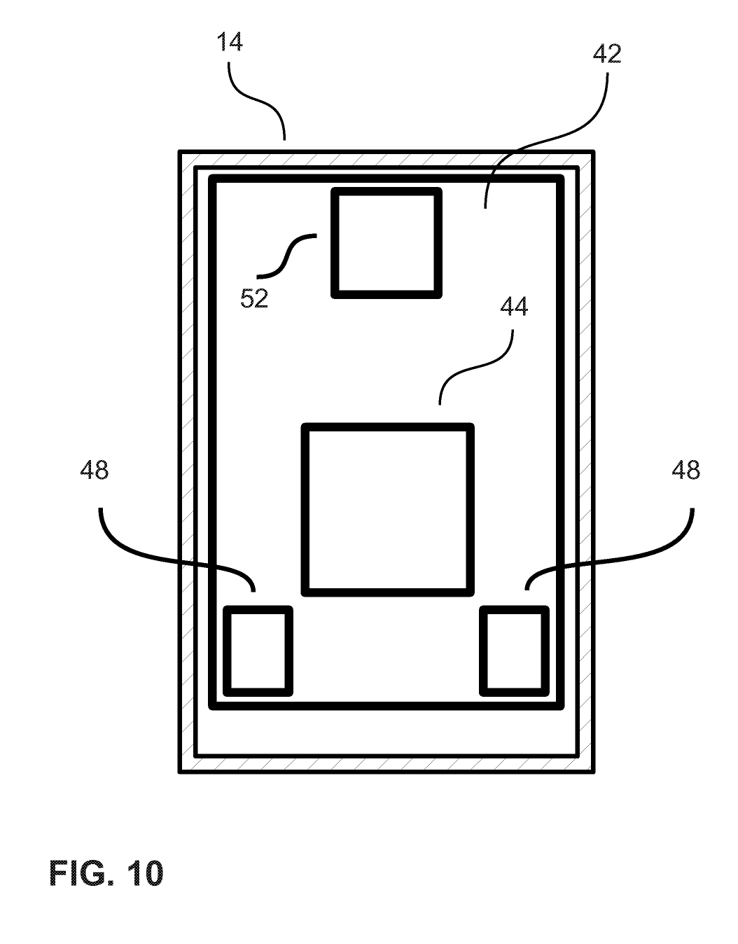

As an alternative means of providing a communicative coupling 16 between the computer 18 and the catheter 10, it is possible to install a miniature Wi-Fi system at the distal end 14. In FIG. 10, which shows the same cross-section through the cap as FIG. 5, a Wi-Fi chip 52, containing a complete Wi-Fi system provides a wireless connection with the computer 18. In this embodiment, it is contemplated to use the NMC1000 chip from NMI of California; however any sufficiently small, low power Wi-Fi chip supporting the IEEE 802.11n standard can also be used.

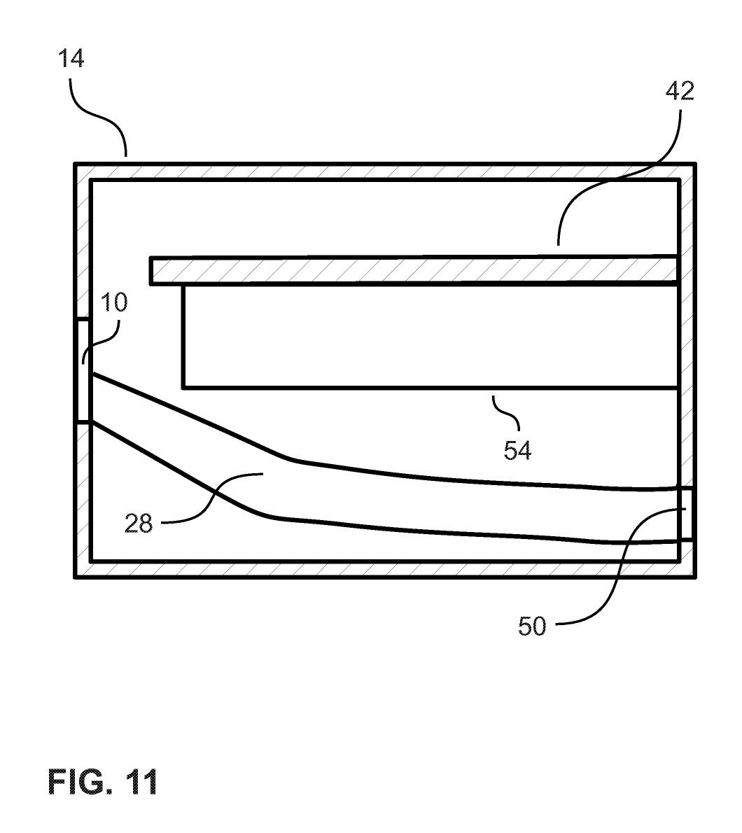

When using a wireless connection, the USB wire is no longer available for powering the sensors and a battery driven power supply is required. In FIG. 11, which shows the same cross-section as in FIG. 6, the USB receptacle 46 has been replaced by a battery driven power supply 54. Battery driven power supplies of this size will not last more than 1 hour under maximum load. Since the surgical procedure itself does not last more than a few minutes once the burr hole 20 is opened, the limited battery life should be sufficient to provide the required navigational assistance procedure described in FIG. 7.

### CONCLUSIONS, RAMIFICATIONS AND SCOPE

Accordingly the reader will see that the embodiments described above provides a number of evident advantages:
(a) The synergistic effect of combining the signals from the video camera, with those from the ultrasonic transducer and the IMU enables the creation of a novel, intelligent guidance system that reduces the likelihood of excess hemorrhaging, while increasing the accuracy of final placement.
(b) The use of an in vivo video camera in the catheter's distal end simplifies the overall system design compared to fiber optic cameras.
(c) The use of an IMU together with the algorithm described in FIG. 9 comprises a means for the system to record the path taken by the catheter en route to the desired location. Information which is invaluable for post-operative diagnostics and treatment.
(d) The use of a portable PC allows the entire system to be carried into the ICU when needed and carried out again when no longer required. Furthermore, the second embodiment with a built-in Wi-Fi system enables the system to operate without the limitations of a wired connection between the parts.

Although the description provided above concerns itself for the most part with the use of the embodiments in the example of a ventriculostomy, those skilled in the art will readily recognize other uses, including, but not limited to, the intracranial placement of catheter's of other types as well as the placement of catheter's in other parts of the body.

It should also be noted, that although the description provided above contains many specific suggestions with regard to the use of components from particular manufactures, the use of particular materials, and the use of particular generic algorithms; these should not be construed as limiting the scope of the embodiments, rather as merely providing illustrative examples of several embodiments.

Therefore the scope of the embodiments should be determined by the appended claims and their legal equivalents, rather than by the examples given above.

What is claimed is:

1. A surgical system, comprising:
(a) a catheter having a predetermined length, a predetermined diameter, a proximal end and a distal end;
(b) a video camera disposed at said distal end;
(c) a first means for illuminating a vicinity of said distal end;
(d) one or more ultrasonic transducers disposed at said distal end;
(e) an inertial measurement unit disposed at said proximal end;
(f) a second means for acquiring and analyzing the plurality of data from said video camera, said ultrasonic transducers and said inertial measurement unit;
(g) a third means for identifying a site in a body;
(h) a fourth means for determining the local position of the catheter relative to a reference point on said body;
(i) a fifth means for providing intelligent navigation assistance to reach said site; and
(j) a sixth means for recording a trajectory of the catheter;
whereby said surgical system increases the safety and accuracy of catheter placement.

2. The surgical system of claim 1 wherein said first means includes a plurality of LED chips disposed at said proximal end, said LED chips being connected to a plurality of fiber optic cables disposed in said catheter and running from said proximal end to said distal end.

3. The surgical system of claim 1 wherein said second means includes a portable computer communicatively coupled to said sensors.

4. The surgical system of claim 1 wherein said third means includes a means for displaying the output from the ultrasonic transducers.

5. The surgical system of claim 1 wherein said fourth means includes a means for determining an orientation of the catheter and a means for determining a depth of insertion.

6. The surgical system of claim 1 wherein said fifth means includes,
(a) a means for displaying output of said video camera;
(b) a means for automatically detecting a blood vessel in said vicinity of said distal end,
(c) a means for automatically determining a new route to said site that does not intersect said blood vessel, and
(d) a means for displaying said new route overlayed upon output from said video camera.

7. The surgical system of claim 1 wherein,
(a) said third means includes a portable computer communicatively coupled to said sensors,
(b) said fourth means includes a means for determining an orientation of the catheter and a means for determining a depth of insertion,
(c) said fifth means includes a means for displaying the output of said video camera, a means for automatically detecting a blood vessel in said vicinity of said distal end, a means for automatically determining a route to said site that does not intersect said blood vessel and a means for displaying said route using augmented reality.

8. A catheter having a predetermined length, a predetermined diameter, a proximal end and a distal end, comprising:
(a) at least one ultrasound transducer disposed in said distal end;
(b) at least one video camera disposed in said distal end;
(c) a plurality of LED chips disposed at said proximal end;
(d) a plurality of fiber optic cables disposed in said catheter, connected to said LED chips and running from said proximal end to said distal end;
(e) at least one inertial measurement unit disposed in said proximal end;
(f) a predetermined number of equally spaced, visible markings running from said distal end to said proximal end;
(g) a drainage canal disposed in said catheter and running from said distal end to said proximal end; and,
(h) one or more devices disposed in said proximal end and electrically connected to said ultrasound transducer, said video camera, said LED chips and said inertial measurement unit.

9. The catheter of claim 8 wherein said one or more devices includes an USB connector.

10. The catheter of claim 8 wherein said one or more devices includes a Wi-Fi system; and a battery operated power supply.

11. A method for a computer assisted navigation to a predetermined site in a body of a catheter having a distal end, a proximal end and equally spaced visible markings running from said distal end to said proximal end, comprising:
(a) disposing a video camera and at least one ultrasonic transducer in said distal end;
(b) disposing an inertial measurement unit in said proximal end;
(c) providing a communicative coupling from said computer to said video camera, said ultrasonic transducer and said inertial measurement unit;
(d) providing a first display means to display a signal from said video camera;
(e) providing a second display means to display the direction the catheter is headed overlayed upon said first display;
(f) providing a third display means to display a signal from said ultrasonic transducer;
(g) providing a first input means for the operator to select said site 100 using said third display means;
(h) determining the depth of insertion from the visible markings;
(i) providing a second input means for entering said depth of insertion;
(j) pushing said catheter into said body;
(k) automatically tracking a trajectory of said catheter and comparing said trajectory to location of said predetermined site;
(l) automatically locating blood vessels in a vicinity of said distal end;
(m) automatically determining a new trajectory to avoid said blood 110 vessels and to reach said predetermined site;
(n) displaying said new trajectory using augmented reality;
(o) issuing a notification when said site is reached; and
(p) recording said trajectory taken by said catheter.
